(19) United States
(12) Patent Application Publication
(10) Pub. No.: US 2024/0398725 A1
(43) Pub. Date: Dec. 5, 2024

(54) NANOPARTICLES FOR CANCER TREATMENT

(71) Applicant: Immix Biopharma, Inc., Los Angeles, CA (US)

(72) Inventors: Ilya Rachman, Los Angeles, CA (US); Gabriel Morris, Los Angeles, CA (US)

(21) Appl. No.: 18/696,333

(22) PCT Filed: Sep. 27, 2022
(86) PCT No.: PCT/US2022/044948

§ 371 Date: Mar. 27, 2024

Related U.S. Application Data
(60) Provisional application No. 63/261,730, filed on Sep. 27, 2021.

(51) Int. Cl. A61K 31/12; A61K 9/107; A61K 31/704; A61K 47/24; A61P 35/00

(52) U.S. Cl. CPC A61K 31/12; A61K 9/1075; A61K 31/704; A61K 47/24; A61P 35/00

(57) ABSTRACT

The invention described herein relates to nanoparticles and micelle constructs comprising a polykinase inhibitor and/or a chemotherapeutic agent and methods of using the same to treat cancer.

### CROSS REFERENCE TO RELATED APPLICATION

This application claims priority to U.S. Provisional Application Ser. No. 63/261,730, filed Sep. 27, 2021, which is hereby incorporated by reference in its entirety.

### TECHNICAL FIELD OF THE INVENTION

The invention disclosed herein generally relates to nanoparticles and compositions containing a polykinase inhibitor and methods of using the same to treat a sarcoma.

### BACKGROUND TO THE INVENTION

Complete and effective treatment for cancer has not been developed despite billions of dollars being spent in cancer research. Part of the reason is because tumor cells can be made up of a variety of cell types, produced as the cells proliferate and incur different mutations. This diversity, in turn, is part of what has made treatment of cancer so difficult, as a population of cancerous cells could easily include a mutant variety that happens to be resistant to any individual treatment or chemotherapy drug that is administered. The few resistant cancer cells are provided a strong selective advantage in comparison to other cells, and over time, those resistant cells increase in frequency.

Thus, there is a need in the art for the development of additional cancer treatments, including those that have the ability to better target drug resistant tumors and potentially bypass the diversity of cancer cells.

### SUMMARY OF INVENTION

Some embodiments of the invention relate to pharmaceutical compositions and methods for treating cancer. In some embodiments, the cancer is sarcoma. In some embodiments, the compositions and methods include a micelle construct and a polykinase inhibitor and/or a chemotherapeutic agent.

In some embodiments, the compositions and methods include a micelle construct including a polykinase inhibitor. In some embodiments, the compositions and methods include a micelle construct including a polykinase inhibitor and a chemotherapeutic agent. In some embodiments, the compositions and methods include both a first micelle construct including a polykinase inhibitor and a chemotherapeutic agent and a second micelle construct including a polykinase inhibitor. In some embodiments, the second micelle construct does not include a chemotherapeutic agent (sometimes referred to herein as “polykinase inhibitor only micelle”).

In some embodiments, the first micelle construct including a polykinase inhibitor and a chemotherapeutic agent and the second micelle construct including a polykinase inhibitor are administered together. In some embodiments, the first micelle construct including a polykinase inhibitor and a chemotherapeutic agent and the second micelle construct including a polykinase inhibitor are administered separately. For example, in some embodiments, the first micelle construct including a polykinase inhibitor and a chemotherapeutic agent and the second micelle construct including a polykinase inhibitor are administered one after the other. In some embodiments, the first micelle construct including a polykinase inhibitor and a chemotherapeutic agent and the second micelle construct including a polykinase inhibitor are administered according to different dosing regimens and/or dosing schedules. For example, in some embodiments, the first micelle construct including a polykinase inhibitor and a chemotherapeutic agent is administered before the second micelle construct including a polykinase inhibitor. In some embodiments, the first micelle construct including a polykinase inhibitor and a chemotherapeutic agent is administered after the second micelle construct including a polykinase inhibitor.

In some embodiments, the polykinase inhibitor is selected from a curcuminoid or curcuminoid analog, derivative or salt thereof or combination thereof. In some embodiments, the wherein the curcuminoid or curcuminoid analog, derivative or salt thereof or combination thereof is a curcumin compound having the structure of formula 1:

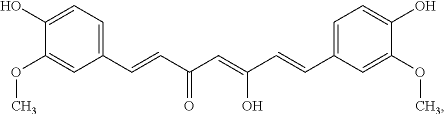 [SMILES: COc1cc(/C=C/C(=O)/C=C(O)/C=C/c2ccc(O)c(OC)c2)ccc1O]

or a curcumin compound having the structure of formula 2:

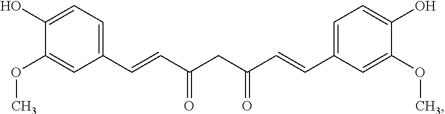 [SMILES: COc1cc(/C=C/C(=O)CC(=O)/C=C/c2ccc(O)c(OC)c2)ccc1O]

In some embodiments, the chemotherapeutic agent is doxorubicin or a pharmaceutical equivalent, analog, derivative, and/or salt thereof.

In some embodiments, the first and second micelle constructs are each formed by amphiphilic PEG2000-DSPE polymers. In some embodiments, the first and second micelle constructs are between 10 nm and 20 nm. In some embodiments, the first and/or second micelle constructs are between 20 nm and 60 nm. In some embodiments, the first and/or second micelle constructs are less than 30 nm. In some embodiments, the first and/or second micelle constructs have an average size from about 1 nm to about 60 nm, or from about 5 nm to about 50 nm, or from about 10 nm to about 40 nm. In some embodiments, the first and/or second micelle constructs have an average size from about 5 nm to about 25 nm, or from about 8 nm to about 22 nm, or from about 12 nm to about 18 nm. In some embodiments, the first and/or second micelle constructs have an average size from about 10 nm to about 20 nm, or from about 12 nm to about 18 nm, or from about 14 nm to about 16 nm. In some embodiments, the first and/or second micelle constructs have an average diameter of less than about 60 nm, or less than about 55 nm, or less than about 50 nm, or less than about 45 nm, or less than about 40 nm, or less than about 35 nm, or less than about 30 nm, or less than about 25 nm, or less than about 20 nm, or less than about 15 nm, or less than about 10 nm. In some embodiments, the first and/or second micelle constructs have an average diameter of about 14 nm, or about 15 nm, or about 16 nm.

In some embodiments, the first and/or second micelle construct comprises a pharmaceutically acceptable carrier.

Some embodiments of the invention relate to methods of treating cancer in a subject. In some embodiments, the cancer is a sarcoma. In some embodiments the cancer type is a surgically unresectable sarcoma. In some embodiments, the sarcoma is a chemotherapy-resistant forms of sarcoma. In some embodiments, the subject is a human. In some embodiments, the methods include administering a therapeutically effective dosage of one or more micelle constructs disclosed herein to the subject.

In some embodiments of the method disclosed herein, administration of the second micelle construct comprising a polykinase inhibitor precedes administration of the first micelle construct comprising a polykinase inhibitor and a chemotherapeutic agent. In some embodiments of the method disclosed herein, administration of the second micelle construct comprising a polykinase inhibitor follows administration of the first micelle construct comprising a polykinase inhibitor and a chemotherapeutic agent.

In some embodiments of the method disclosed herein, the second micelle construct comprising a polykinase inhibitor is administered once per day. In some embodiments of the method disclosed herein, the second micelle construct comprising a polykinase inhibitor is administered twice per day. In some embodiments of the method disclosed herein, the second micelle construct comprising a polykinase inhibitor is administered three times per day.

In some embodiments, the method further includes administering the second micelle construct comprising a polykinase inhibitor for up to 14 days after completion of the regimen of claim 1. In some embodiments, the method further includes administering the second micelle construct comprising a polykinase inhibitor for 15 to 28 days after completion of the regimen of claim 1. In some embodiments, the method further includes administering the second micelle construct comprising a polykinase inhibitor for 28 days or more after completion of the regimen of claim 1.

In some embodiments of the method disclosed herein, the micelle construct comprising a polykinase inhibitor is administered at a dosage of about 10 mg/m²/day to about 40 mg/m²/day, or at about 20 mg/m²/day to about 200 mg/m² per day, or at about 40 mg/m²/day to about 200 mg/m²/day, or at about 50 mg/m²/day to about 175 mg/m² per day, or at about 75 mg/m²/day to about 150 mg/m² per day, or at about 100 mg/m²/day to about 150 mg/m² per day, or at about 20 mg/m²/day to about 100 mg/m² per day, or at about 25 mg/m²/day to about 75 mg/m² per day, or at about 50 mg/m²/day to about 150 mg/m² per day, or at about 100 mg/m²/day to about 200 mg/m² per day, or at about 200 mg/m² to about 1000 mg/m² per week.

Some embodiments of the invention relate to methods of inhibiting cell growth of a tumor cell. In some embodiments, the methods include administering a therapeutically effective does of one or more micelle constructs disclosed herein to a tumor cell. In some embodiments of the method, administration of the second micelle construct comprising a polykinase inhibitor precedes administration of the first micelle construct comprising a polykinase inhibitor and a chemotherapeutic agent. In some embodiments, administration of the second micelle construct comprising a polykinase inhibitor follows administration of the first micelle construct comprising a polykinase inhibitor and a chemotherapeutic agent.

In some embodiments of the method disclosed herein, the second micelle construct comprising a polykinase inhibitor is administered once per day. In some embodiments of the method disclosed herein, the second micelle construct comprising a polykinase inhibitor is administered twice per day. In some embodiments of the method disclosed herein, the second micelle construct comprising a polykinase inhibitor is administered three times per day.

In some embodiments, the method further includes administering the second micelle construct comprising a polykinase inhibitor for up to 14 days after completion of the regimen of claim 1. In some embodiments, the method further includes administering the second micelle construct comprising a polykinase inhibitor for 15 to 28 days after completion of the regimen of claim 1. In some embodiments, the method further includes administering the second micelle construct comprising a polykinase inhibitor for 28 days or more after completion of the regimen of claim 1.

In some embodiments of the method disclosed herein, the micelle construct comprising a polykinase inhibitor is administered at a dosage of about 10 mg/m²/day to about 40 mg/m²/day, or at about 20 mg/m²/day to about 200 mg/m² per day, or at about 40 mg/m²/day to about 200 mg/m²/day, or at about 50 mg/m²/day to about 175 mg/m² per day, or at about 75 mg/m²/day to about 150 mg/m² per day, or at about 100 mg/m²/day to about 150 mg/m² per day, or at about 20 mg/m²/day to about 100 mg/m² per day, or at about 25 mg/m²/day to about 75 mg/m² per day, or at about 50 mg/m²/day to about 150 mg/m² per day, or at about 100 mg/m²/day to about 200 mg/m² per day, or at about 200 mg/m² to about 1000 mg/m² per week.

### BRIEF DESCRIPTION OF THE DRAWINGS

FIGS. 1A and 1B are graphs depicting the effect of curcumin plus doxorubicin micelles on tumor size in STS Xenograft Model (81 days). FIG. 1A shows tumor sizes in mm³. Note, where n=1, no SEM bars are shown. Curcumin plus doxorubicin micelles has SEM bars from days 0-65. From days 0-18, curcumin plus doxorubicin micelles bars are less than 30 mm³ (too small to be visible). FIG. 1B shoes tumor size percentage change from baseline. Note, where n=1, no SEM bars are shown. Curcumin plus doxorubicin micelles has SEM bars from days 0-65. From days 0-18, curcumin plus doxorubicin micelles SEM bars are less than 20% (too small to be visible).

FIG. 2 is a graph depicting the effect of curcumin plus doxorubicin micelles on survival in STS Xenograft Model (81 days).

FIG. 3 is a graph depicting the effect of curcumin plus doxorubicin micelles plus Curcumin-only micelle on tumor growth in STS Xenograft Model.

FIG. 4 is a graph depicting the effects of additional curcumin-only micelles and the duration of treatment in a xenograft model of sarcoma HT1080.

FIG. 5 is a graph depicting the effects of additional curcumin-only micelles and the dosing schedules in a xenograft model of sarcoma HT1080.

FIG. 6 is a graph depicting the effects of increasing the number of daily doses of polykinase inhibitor curcumin micelle in a syngeneic mouse sarcoma model.

FIG. 7 is a graph depicting the effects of extending the treatment duration with polykinase inhibitor curcumin micelle in a syngeneic mouse sarcoma model S180.

### DETAILS OF INVENTION

Embodiments of the invention disclosed herein provide a novel pharmaceutical regimen comprising a combination of a first nanoparticle composition comprising a micelle co-loaded with a kinase inhibitor and a chemotherapeutic or chemotherapy agent and a second nanoparticle composition comprising a micelle and a polykinase inhibitor. The examples described herein demonstrate that the compositions and methods disclosed herein result in a superior cancer killing effect via inducing a greater amount of apoptosis in cancer cells than a sum of individual effects of these agents administered separately. The data provided in the Examples demonstrate efficacy of this therapeutic construct in doxorubicin-resistant sarcoma xenografts as well as in a sarcoma syngeneic model.

Embodiments of the invention disclosed herein relate to the novel and unexpected finding that it is the polykinase concentration and the frequency of its administration that predominantly determines the anti-tumor efficacy of this regimen. For example, curcumin plus doxorubicin micelles alone showed potent efficacy in a Dox-resistant sarcoma model, with further surprising improvement in efficacy and survival seen with the addition of Cur-containing micelle construct to curcumin plus doxorubicin micelles (resulting in an increased Cur:Dox ratio), without any added toxicity. Thus, curcumin plus doxorubicin micelles with added Cur-containing nanoparticles is an effective treatment option for patients with difficult-to-treat cancers that are typically resistant to other drugs, such as sarcoma.

Furthermore, embodiments of the invention disclosed herein relate to the newly discovered range of effective concentrations and schedules of administration used for nanoparticles encapsulating a curcuminoid complex and doxorubicin. Further information can be found in U.S. Patent Publication No. US 2020-0179282 A1 and International Patent Application Serial No. PCT/US2022/036419, each of which is fully incorporated by reference in its entirety.

All references, publications, and patents cited herein are incorporated by reference in their entirety as though they are fully set forth. Unless defined otherwise, technical and scientific terms used herein have the same meaning as commonly understood by one of ordinary skill in the art to which this invention belongs. Hornyak, et al., Introduction to Nanoscience and Nanotechnology, CRC Press (2008); Singleton et al., Dictionary of Microbiology and Molecular Biology 3rd ed., J. Wiley & Sons (New York, NY 2001); March, Advanced Organic Chemistry Reactions, Mechanisms and Structure 7th ed., J. Wiley & Sons (New York, NY 2013); and Sambrook and Russel, Molecular Cloning: A Laboratory Manual 4th ed., Cold Spring Harbor Laboratory Press (Cold Spring Harbor, NY 2012), provide one skilled in the art with a general guide to many of the terms used in the disclosure herein. One skilled in the art will recognize many methods and materials similar or equivalent to those described herein, which could be used in the practice of the present invention. Indeed, the embodiments of the invention disclosed herein in no way limited to the methods and materials described.

### Nanoparticles

The nanoparticles of embodiments of the invention disclosed herein can be liposomes that include an aqueous compartment enclosed by at least one lipid bilayer. When lipids that include a hydrophilic headgroup are dispersed in water, they spontaneously form bilayer membranes referred to as lamellae. The lamellae are composed of two monolayer sheets of lipid molecules with their non-polar (hydrophobic) surfaces facing each other and their polar (hydrophilic) surfaces facing the aqueous medium.

In some embodiments, the nanoparticles of the regimen disclosed herein have a size from about 1 nm to about 100 nm. In some embodiments, the nanoparticles disclosed herein have a size of about 1 nm, or about 5 nm, or about 10 nm, or about 15 nm, or about 20 nm, or about 25 nm, or about 30 nm, or about 35 nm, or about 40 nm, or about 45 nm, or about 50 nm, or about 55 nm, or about 60 nm, or about 65 nm, or about 70 nm, or about 75 nm, or about 80 nm, or about 85 nm, or about 90 nm, or about 95 nm, or about 100 nm. In some embodiments, the nanoparticles of the regimen disclosed herein have an average diameter of less than about 50 nm, or less than about 45 nm, or less than about 40 nm, or less than about 35 nm, or less than about 30 nm, or less than about 25 nm, or less than about 20 nm, or less than about 18 nm, or less than about 16 nm, or less than about 15 nm, or less than about 14 nm, or less than about 12 nm, or less than about 10 nm.

In some embodiments, the nanoparticles of the regimen disclosed herein are between about 10 nm and 60 nm, or between about 20 nm to about 50 nm, or between about 25 nm to about 40 nm. In some embodiments, the nanoparticles disclosed herein are between about 1 nm and 50 nm, or between about 5 nm to about 40 nm, or between about 10 nm to about 30 nm. In some embodiments, the nanoparticles disclosed herein are between about 10 nm and about 20 nm, or between about 12 nm and about 18 nm, or between about 14 nm and about 16 nm.

In some embodiments, the nanoparticles disclosed herein are micelle constructs that include amphiphilic polymers with a hydrophilic exterior and a hydrophobic interior compartment. When these amphiphilic polymers are exposed to an aqueous environment, they spontaneously assemble into single layer complexes with their non-polar hydrophobic portions facing the interior core of the nanoparticle.

In some embodiments, the micelle constructs disclosed herein have a size from about 1 nm to about 60 nm, or from about 5 nm to about 50 nm, or from about 10 nm to about 40 nm. In some embodiments, the micelle constructs disclosed herein have a size from about 5 nm to about 25 nm, or from about 8 nm to about 22 nm, or from about 12 nm to about 18 nm. In some embodiments, the micelle constructs disclosed herein have a size from about 10 nm to about 20 nm, or from about 12 nm to about 18 nm, or from about 14 nm to about 16 nm. In some embodiments, the micelle constructs disclosed herein have an average diameter of less than about 60 nm, or less than about 55 nm, or less than about 50 nm, or less than about 45 nm, or less than about 40 nm, or less than about 35 nm, or less than about 30 nm, or less than about 25 nm, or less than about 20 nm, or less than about 15 nm, or less than about 10 nm. In some embodiments, the micelle constructs disclosed herein have an average diameter of about 14 nm, or about 15 nm, or about 16 nm.

### Lipids

In some embodiments, the nanoparticles provided herein include a lipid. Suitable lipids can include fats, waxes, steroids, cholesterol, fat-soluble vitamins, monoglycerides, diglycerides, phospholipids, sphingolipids, glycolipids, cationic or anionic lipids, derivatized lipids, and the like.

Suitable phospholipids include, but are not limited to, phosphatidylcholine (PC), phosphatidic acid (PA), phosphatidylethanolamine (PE), phosphatidylglycerol (PG), phosphatidylserine (PS), and phosphatidylinositol (PI), dimyristoyl phosphatidyl choline (DMPC), distearoyl phosphatidyl choline (DSPC), dioleoyl phosphatidyl choline (DOPC), dipalmitoyl phosphatidyl choline (DPPC), dimyristoyl phosphatidyl glycerol (DMPG), distearoyl phosphatidyl glycerol (DSPG), dioleoyl phosphatidyl glycerol (DOPG), dipalmitoyl phosphatidyl glycerol (DPPG), dimyristoyl phosphatidyl serine (DMPS), distearoyl phosphatidyl serine (DSPS), dioleoyl phosphatidyl serine (DOPS), dipalmitoyl phosphatidyl serine (DPPS), dioleoyl phosphatidyl ethanolamine (DOPE), palmitoyloleoylphosphatidylcholine (POPC), palmitoyloleoyl-phosphatidylethanolamine (POPE) and dioleoyl-phosphatidylethanolamine 4-(Nmaleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearoyl-2-oleoyl-phosphatidyethanolamine (SOPE), 1,2-dielaidoyl-sn-glycero-3-phophoethanolamine (transDOPE), and cardiolipin.

In some embodiments, the lipids include derivatized lipids, such as PEGylated lipids. Derivatized lipids include, for example, DSPE-PEG2000, cholesterol-PEG2000, DSPE-polyglycerol, or other derivatives generally known in the art. In some embodiments, the lipid is DSPE-PEG2000. In some embodiments, the micelle construct is formed by amphiphilic PEG2000-DSPE polymers.

### Kinase Inhibitors

In some embodiments, the micelle constructs disclosed herein include a hydrophobic kinase inhibitor. The hydrophobic kinase inhibitor can be polykinase inhibitor. In some embodiments, the polykinase inhibitor is a curcuminoid or curcuminoid analog, derivative or salt thereof. In some embodiments, the inhibitor can be EF24, EF31 and other compounds disclosed in U.S. Pat. No. 7,842,705, which is hereby incorporated by reference in its entirety. In some embodiments, the nanoparticles disclosed herein include a combination of different kinase inhibitors. The kinase inhibitors can be natural or synthetic. In some embodiments, the kinase inhibitor is an NF-kb or Stat3 or poly-kinase inhibitor.

In some embodiments, the kinase inhibitor is a polyphenolic kinase inhibitor. In some embodiments, the polyphenolic kinase inhibitor is a polyphenol curcuminoid complex (PCC). Curcuminoids are polyphenolic pigments and include curcumin, demethoxycurcumin, and bisdemethoxycurcumin. In some embodiments, the kinase inhibitor is curcumin, or a derivative of curcumin, or a curcumin analogue, or a curcumin metabolite. In some embodiments, the kinase inhibitor is a synthetic analog of curcumin.

As used herein curcumin is also known as diferuloylmethane or (E,E)-1,7-bis (4 hydroxy-3-methoxyphenyl)-1,6-heptadiene-3,5,-dione. Curcumin can be derived from a natural source, the perennial herb Curcuma longa L., which is a member of the Zingiberaceae family.

Curcumin is soluble in ethanol, alkalis, ketones, acetic acid and chloroform. Curcumin is insoluble in water. Curcumin is therefore lipophilic, and generally readily associates with lipids, e.g., many of those used in the colloidal drug-delivery systems of embodiments of the invention disclosed herein. In some embodiments, curcumin is formulated as a metal chelate.

As used herein, curcumin analogues are those compounds which due to their structural similarity to curcumin, exhibit effects similar to that of curcumin. Curcumin analogues include, but are not limited, to Ar-tumerone, methylcurcumin, demethoxy curcumin, bisdemethoxycurcumin, sodium curcuminate, dibenzoylmethane, acetylcurcumin, feruloyl methane, tetrahydrocurcumin, 1,7-bis(4-hydroxy-3-methoxyphenyl)-1,6-heptadiene-3,5-dione (curcumin1), 1,7-bis(piperonyl)-1,6-heptadiene-3,5-dione (piperonyl curcumin) 1,7-bis(2-hydroxy naphthyl)-1,6-heptadiene-2,5-dione (2-hydroxyl naphthyl curcumin), 1,1-bis (phenyl)-1,3,8,10 undecatetraene-5,7-dione (cinnamyl curcumin) and the like. Curcumin analogues also include isomers of curcumin, such as the (Z,E) and (Z,Z) isomers of curcumin.

In some embodiments, curcumin metabolites can also be used. Known curcumin metabolites include glucoronides of tetrahydrocurcumin and hexahydrocurcumin, and dihydroferulic acid. In some embodiments, curcumin analogues or metabolites are formulated as metal chelates, especially copper chelates. Other appropriate derivatives of curcumin, curcumin analogues and curcumin metabolites appropriate for use in embodiments of the invention disclosed herein will be apparent to one of skill in the art.

In some embodiments, the curcumin is selected from the group consisting of Ar-tumerone, methylcurcumin, demethoxy curcumin, bisdemethoxycurcumin, sodium curcuminate, dibenzoylmethane, acetylcurcumin, feruloyl methane, tetrahydrocurcumin, 1,7-bis (4-hydroxy-3-methoxyphenyl)-1,6-heptadiene-3,5-dione (curcumin1), 1,7-bis(piperonyl)-1,6-heptadiene-3,5-dione (piperonyl curcumin) 1,7-bis(2-hydroxy naphthyl)-1,6-heptadiene-2,5-dione (2-hydroxyl naphthyl curcumin) and 1,1-bis(phenyl)-1,3,8,10 undecatetraene-5,7-dione.

In some embodiments, the kinase inhibitor is a compound having the structure of formula 1:

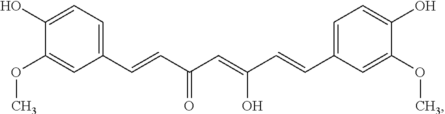 [SMILES: COc1cc(/C=C/C(=O)/C=C(O)/C=C/c2ccc(O)c(OC)c2)ccc1O]

or a compound having the structure of formula 2:

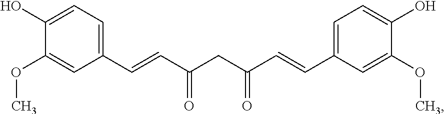 [SMILES: COc1cc(/C=C/C(=O)CC(=O)/C=C/c2ccc(O)c(OC)c2)ccc1O]

### Chemotherapeutic Agents

In some embodiments, the nanoparticles disclosed herein optionally include one or more chemotherapeutic or chemotherapy agents. Exemplary chemotherapeutic agents include, but are not limited to, an anthracycline (e.g., doxorubicin), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, tositumomab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide). In some embodiments, the chemotherapeutic agent is an inducer of apoptosis. In some embodiments, the chemotherapeutic agent is a PEG-PE doxorubicin complex. In some embodiments, the micelle construct is formed by amphiphilic PEG2000-DSPE polymers.

In some embodiments, the nanoparticle disclosed herein is micelle construct co-loaded with a polykinase inhibitor and doxorubicin.

In some embodiments, the nanoparticle disclosed herein is a micelle construct of a curcuminoid complex co-loaded with doxorubicin.

### Therapeutic Compositions

In some embodiments, therapeutic compositions including the nanoparticles or micelle constructs described herein are provided. The therapeutic compositions can further include, for example, a pharmaceutically acceptable carrier and, optionally, other desired components, including, but not limited to, stabilizers, preservatives, fillers, and the like. In some embodiments, the carrier(s) are acceptable in the sense of being compatible with the other ingredients of the formula and not deleterious to the recipient thereof. Selection of appropriate carriers, e.g., phosphate buffered saline and the like, are well within the skill of those in the art. Similarly, one skilled in the art can readily select appropriate stabilizers, preservatives, and the like for inclusion in the composition.

Any route of administration known in the art can be employed for administration of the nanoparticle, e.g., subcutaneous, intraperitoneal, intravenous (i.v.), intramuscular (i.m.), intrasternal, intratumoral, infusion, oral, intramuscular, intranasal and the like. In some embodiments, the therapeutic compositions disclosed herein are suitable for delivery by i.v. administration.

### Methods of Making the Compositions

In some embodiments, methods of producing the nanoparticles and/or micelle constructs described herein are provided. In some embodiments, the methods include mixing a phospholipid, a polyphenolic kinase inhibitor (such, as a curcuminoid) or other hydrophobic kinase inhibitor with an organic solvent to solubilize the mixture. If a chemotherapeutic agent is used, the agent is included in the mixture.

In some embodiments, after mixing, the solvent is evaporated by published methods and the mixture is rehydrated in PBS. The physicochemical properties, such as particle size, surface charge, the encapsulation efficiency and content can be determined according to published methods. Further information can be found in Sarisozen et. al. European Journal of Pharmaceutics and Biopharmaceutics 108 (2016) 54-67, which is hereby incorporated by reference in its entirety.

### Methods of Using the Compositions

In some embodiments, methods of using the regimen and/or nanoparticles and/or micelle constructs disclosed herein are provided. In some embodiments, the method of using relates to treating cancer in a subject.

The term “cancer” refers to a disease characterized by the rapid and uncontrolled growth of aberrant cells. Cancer cells can spread locally or through the bloodstream and lymphatic system to other parts of the body. Examples of cancers used in the invention include but are not limited to, Acute Lymphoblastic Leukemia (ALL), Acute Myeloid Leukemia (AML), Adrenocortical Carcinoma, Anal Cancer, Appendix Cancer, Atypical Teratoid/Rhabdoid Tumor, Basal Cell Carcinoma, Bile Duct Cancer, Bladder Cancer, Bone Cancer, Brain Tumor, Astrocytoma, Brain and Spinal Cord Tumor, Brain Stem Glioma, Central Nervous System Atypical Teratoid/Rhabdoid Tumor, Central Nervous System Embryonal Tumors, Breast Cancer, Bronchial Tumors, Burkitt Lymphoma, Carcinoid Tumor, Carcinoma of Unknown Primary, Central Nervous System Cancer, Cervical Cancer, Childhood Cancers, Chordoma, Chronic Lymphocytic Leukemia (CLL), Chronic Myelogenous Leukemia (CML), Chronic Myeloproliferative Disorders, Colon Cancer, Colorectal Cancer, Craniopharyngioma, Cutaneous T-Cell Lymphoma, Ductal Carcinoma In Situ (DCIS), Embryonal Tumors, Endometrial Cancer, Ependymoblastoma, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer, Fibrous Histiocytoma of Bone, Gallbladder Cancer, Gastric Cancer, Gastrointestinal Carcinoid Tumor, Gastrointestinal Stromal Tumors (GIST), Germ Cell Tumor, Ovarian Germ Cell Tumor, Gestational Trophoblastic Tumor, Glioma, Hairy Cell Leukemia, Head and Neck (Nasopharyngeal) Cancer, Heart Cancer, Hepatocellular Cancer, Histiocytosis, Langerhans Cell Cancer, Hodgkin Lymphoma, Hypopharyngeal Cancer, Intraocular Melanoma, Islet Cell Tumors, Kaposi Sarcoma, Kidney Cancer, Langerhans Cell Histiocytosis, Laryngeal Cancer, Leukemia, Lip and Oral Cavity Cancer, Liver Cancer, Lobular Carcinoma In Situ (LCIS), Lung Cancer, Lymphoma, AIDS-Related Lymphoma, Macroglobulinemia, Male Breast Cancer, Medulloblastoma, Medulloepithelioma, Melanoma, Merkel Cell Carcinoma, Malignant Mesothelioma, Metastatic Squamous Neck Cancer with Occult Primary, Midline Tract Carcinoma Involving NUT Gene, Mouth Cancer, Multiple Endocrine Neoplasia Syndrome, Multiple Myeloma/Plasma Cell Neoplasm, Mycosis Fungoides, Myelodysplastic Syndrome, Myelodysplastic/Myeloproliferative Neoplasm, Chronic Myelogenous Leukemia (CIVIL), Acute Myeloid Leukemia (AML), Myeloma, Multiple Myeloma, Chronic Myeloproliferative Disorder, Nasal Cavity Cancer, Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip Cancer, Oropharyngeal Cancer, Osteosarcoma, Ovarian Cancer, Pancreatic Cancer, Papillomatosis, Paraganglioma, Paranasal Sinus Cancer, Nasal Cavity Cancer, Parathyroid Cancer, Penile Cancer, Pharyngeal Cancer, Pheochromocytoma, Pineal Parenchymal Tumors of Intermediate Differentiation, Pineoblastoma, Pituitary Tumor, Plasma Cell Neoplasm, Pleuropulmonary Blastoma, Breast Cancer, Primary Central Nervous System (CNS) Lymphoma, Prostate Cancer, Rectal Cancer, Renal Cell Cancer, Clear cell renal cell carcinoma, Renal Pelvis Cancer, Ureter Cancer, Transitional Cell Cancer, Retinoblastoma, Rhabdomyosarcoma, Salivary Gland Cancer, Sarcoma, Sezary Syndrome, Skin Cancer, Small Cell Lung Cancer, Small Intestine Cancer, Soft Tissue Sarcoma, Squamous Cell Carcinoma, Squamous Neck Cancer with Occult Primary, Squamous Cell Carcinoma of the Head and Neck (HNSCC), Stomach Cancer, Supratentorial Primitive Neuroectodermal Tumors, T-Cell Lymphoma, Testicular Cancer, Throat Cancer, Thymoma, Thymic Carcinoma, Thyroid Cancer, Transitional Cell Cancer of the Renal Pelvis and Ureter, Triple Negative Breast Cancer (TNBC), Gestational Trophoblastic Tumor, Unknown Primary, Unusual Cancer of Childhood, Urethral Cancer, Uterine Cancer, Uterine Sarcoma, Waldenström Macroglobulinemia, Wilms Tumor, and the like. In some embodiments, the cancer is sarcoma.

In some embodiments, the regimen disclosed herein includes a first nanoparticle comprising a micelle construct, a polykinase inhibitor, and a chemotherapeutic agent, and a second nanoparticle comprising a micelle construct and a polykinase inhibitor without a chemotherapeutic agent. In some embodiments, the methods of using the regimen include administering a therapeutically effective amount of the first nanoparticle and administering a therapeutically effective amount of the second nanoparticle.

The term “effective amount” or “therapeutically effective amount” are used interchangeably herein, and refer to an amount of a compound, formulation, material, or composition, as described herein effective to achieve a particular biological result.

In some embodiments, the methods disclosed herein include administration of a composition comprising the nanoparticle or micelle construct to a subject. In some embodiments, administration can be intravenous, oral, inhaled, intranasal, rectal, topical, and the like.

In some embodiments, the methods disclosed herein include administering a nanoparticle comprising a micelle construct and a polykinase inhibitor without a chemotherapeutic agent from several minutes to eight to twelve hours before administering a nanoparticle comprising a micelle construct, a polykinase inhibitor, and a chemotherapeutic agent. In some embodiments, the regimen includes administering a nanoparticle comprising a micelle construct and a polykinase inhibitor without a chemotherapeutic agent up to eight hours after administering a nanoparticle comprising a micelle construct, a polykinase inhibitor, and a chemotherapeutic agent.

In some embodiments, the methods disclosed herein include dosing the first and/or second nanoparticle compositions disclosed herein once a day, twice a day, three times per day, or about every 2 hours, or 3 hours, or 4 hours, or 5 hours, or 6 hours, or 7 hours, or 8 hours, or 12 hours, or 24 hours. In some embodiments, the regimen treatment length is from about 5 days to about 28 days. For example, the treatment length can be five days, 10 days, or five days for two weeks, or five days for three weeks, or five days for four weeks, or more.

In some embodiments, the methods disclosed herein further include administering additional and/or higher doses of a nanoparticle comprising a micelle construct and a polykinase inhibitor without a chemotherapeutic agent, such as about 22 mg/kg/dose to about 30 mg/kg/dose, or about 30 mg/kg/dose to about 50 mg/kg/dose, or about 50 mg/kg/dose to about 100 mg/kg/dose of a nanoparticle comprising a polykinase inhibitor without a chemotherapeutic agent.

In some embodiments, the nanoparticle comprising a micelle construct and a polykinase inhibitor without a chemotherapeutic agent is administered once per day. In some embodiments, the nanoparticle comprising a micelle construct and a polykinase inhibitor without a chemotherapeutic agent is administered twice per day. In some embodiments, the nanoparticle comprising a micelle construct and polykinase inhibitor without a chemotherapeutic agent is administered three times per day.

In some embodiments, the regimen further includes administering a nanoparticle comprising a micelle construct and a polykinase inhibitor without a chemotherapeutic agent for up to fourteen (14) days after completion of the final dose of the nanoparticle comprising a micelle construct, a polykinase inhibitor and a chemotherapeutic agent. In some embodiments, the regimen further includes administering a nanoparticle comprising a micelle construct and a polykinase inhibitor without a chemotherapeutic agent for fifteen (15) to twenty-eight (28) days after completion of the final dose of the nanoparticle comprising a micelle construct, polykinase inhibitor and a chemotherapeutic agent. In some embodiments, the regimen further includes administering a nanoparticle comprising a micelle construct and a polykinase inhibitor without a chemotherapeutic agent for twenty-eight (28) days or more after completion of the final dose of the nanoparticle comprising a micelle construct, polykinase inhibitor and a chemotherapeutic agent.

In some embodiments, the polykinase inhibitor is selected from a curcuminoid or curcuminoid analog, derivative or salt thereof or combination thereof. In some embodiments, the curcuminoid is a curcumin compound. In some embodiments, the chemotherapeutic agent is doxorubicin or a pharmaceutical equivalent, analog, derivative, and/or salt thereof. In some embodiments, the micelle construct is formed by amphiphilic PEG2000-DSPE polymers. In some embodiments, the micelle construct is between about 10 nm and about 20 nm, or between about 12 nm and about 18 nm, or between about 14 nm and about 16 nm.

In some embodiments, the nanoparticles or micelle constructs disclosed herein include doxorubicin and/or curcumin and dosing in humans is performed intravenously (IV) as follows. In some embodiments, doxorubicin is administered at about 0.5 mg/m²/day to 15 mg/m²/day, or at about 1 mg/m²/day to 12.5 mg/m²/day, or at about 2 mg/m²/day to 12 mg/m²/day, or at about 2.5 mg/m²/day to 10 mg/m²/day. For example, in some embodiments, doxorubicin is administered at about 1 mg/m²/day, or about 1.5 mg/m²/day, or about 2 mg/m²/day, or about 2.5 mg/m²/day, or about 3 mg/m²/day, or about 3.5 mg/m²/day, or about 4 mg/m²/day, or about 4.5 mg/m²/day, or about 5 mg/m²/day, or about 6 mg/m²/day, or about 7 mg/m²/day, or about 8 mg/m²/day, or about 9 mg/m²/day, or about 10 mg/m²/day, or about 11 mg/m²/day, or about 12 mg/m²/day, or about 13 mg/m²/day, or about 14 mg/m²/day, or about 15 or more mg/m²/day.

In some embodiments, doxorubicin is administered at about 5 mg/m² per week, or at about 6 mg/m² per week, or at about 7 mg/m² per week, or at about 8 mg/m² per week, or at about 9 mg/m² per week, or at about 10 mg/m² per week, or at about 12 mg/m² per week, or at about 15 mg/m² per week, or at about 20 mg/m² per week, or at about 25 mg/m² per week, or at about 30 mg/m² per week, or at about 40 mg/m² per week, or at about 50 mg/m² per week, or at about 60 mg/m² per week, or at about 70 mg/m² per week.

In some embodiments, doxorubicin is administered at about 5 mg/m² weekly, or about 6 mg/m² weekly, or about 7 mg/m² weekly, or about 8 mg/m² weekly, or about 9 mg/m² weekly, or about 10 mg/m² weekly, or about 11 mg/m² weekly, or about 12 mg/m² weekly, or about 13 mg/m² weekly, or about 14 mg/m² weekly, or about 15 or more mg/m² weekly.

In some embodiments, curcumin is administered at about 10 mg/m²/day to about 40 mg/m²/day, or at about 20 mg/m²/day to about 200 mg/m² per day, or at about 40 mg/m²/day to about 200 mg/m²/day, or at about 50 mg/m²/day to about 175 mg/m² per day, or at about 75 mg/m²/day to about 150 mg/m² per day, or at about 100 mg/m²/day to about 150 mg/m² per day, or at about 20 mg/m²/day to about 100 mg/m² per day, or at about 25 mg/m²/day to about 75 mg/m² per day, or at about 50 mg/m²/day to about 150 mg/m² per day, or at about 100 mg/m²/day to about 200 mg/m² per day, or at about 200 mg/m² to about 1000 mg/m² per week.

For example, in some embodiments, curcumin is administered at about 10 mg/m²/day, or about 15 mg/m²/day, or about 20 mg/m²/day, or about 25 mg/m²/day, or about 30 mg/m²/day, or about 35 mg/m²/day, or about 40 mg/m²/day, or about 50 mg/m²/day, or about 60 mg/m²/day, or about 70 mg/m²/day, or about 80 mg/m²/day, or about 90 mg/m²/day, or about 100 mg/m²/day, or about 125 mg/m²/day, or about 150 mg/m²/day, or about 175 mg/m²/day, or about 200 or more mg/m²/day.

In some embodiments, curcumin is administered at about 70 mg/m² per week, or about 80 mg/m² per week, or about 90 mg/m² per week, or about 100 mg/m² per week, or about 125 mg/m² per week, or about 150 mg/m² per week, or about 175 mg/m² per week, or about 200 mg/m² per week, or about 300 mg/m² per week, or about 400 mg/m² per week, or about 500 mg/m² per week, or about 600 mg/m² per week, or about 700 mg/m² per week, or about 800 mg/m² per week, or about 900 mg/m² per week, or about 1000 mg/m² per week, or about 1100 mg/m² per week, or about 1200 mg/m² per week, or about 1300 mg/m² per week, or about 1400 or more mg/m² per week, or about 1500 or more mg/m² per week, or about 1600 or more mg/m² per week, or about 1700 or more mg/m² per week, or about 1800 or more mg/m² per week, or about 1900 or more mg/m² per week, or about 1200 or more mg/m² per week, or about 2100 or more mg/m² per week.

In mice, the dosing range can be as follows: doxorubicin can be administered at about 1.5 mg/kg/day to 2.5 mg/kg/day or 8 mg/kg weekly; and curcumin can be administered at 11 mg/kg/day to 32 mg/kg/day×5 doses, or 32 to 140 mg/kg/day, or 72 mg/kg to 220 mg/kg per week.

### EXAMPLES

### Example 1

### Mouse Xenograft Model of Dox-Resistant Soft Tissue Sarcoma

Curcumin plus doxorubicin micelles was evaluated in an STS doxorubicin-resistant xenograft mouse model (SW872-DXR). The dedifferentiated liposarcoma cell line (SW872) is one of the earliest STS model systems that has been extensively studied in preclinical investigations (Dodd 2010, Stratford 2012).

The SW872-DXR model was derived from the commercial cell line SW872. The resistance to doxorubicin was initiated in 2D cultures, by adding increased concentrations of doxorubicin every 2 weeks, starting from the IC₅₀ concentrations of doxorubicin of the “parental” cell line SW872. The resistance of this cell line was verified by 3-(4,5-dimethylthiazol-2-yl)-5-(3-carboxymethoxyphenyl)-2-(4-sulfophenyl)-2H-tetrazolium (MTS) assay. IC₅₀ of doxorubicin in this model increased from +/−200 nM to >1 μM.

The SW872 xenograft model was generated by grafting tumor specimens (around 3 mm³) subcutaneously (SC) into the right flank of 6 to 8 week-old female Hsd:Athymic Nude-Foxn1^{nu} nude mice to develop highly proliferative tumors. The growth of SC tumors was followed by biweekly measurement of tumor diameters with a Vernier caliper and tumor volume (TV) calculated according to the formula:

$$TV ( {mm}^{3} ) = d 2 \times D / 2$$

where “d” and “D” were the shortest and the longest diameter, respectively.

Mice (n=8) were divided into the following treatment groups: control treated with PBS buffer only (83 μl/dose, n=1); empty polyethylene glycol-phosphatidyl ethanolamine (PEG-PE) micelles (83 μl/dose, n=2); doxorubicin-only micelles (2.5 mg/kg dose×doses, n=4), curcumin-only micelles (11 mg/kg dose×5 doses, n=4); curcumin plus doxorubicin micelles (2.5 mg/kg/doxorubicin dose×5 doses, n=4) and curcumin-only micelles (21.7 mg/kg curcumin dose) followed by curcumin plus doxorubicin micelles (2.5 mg/kg/doxorubicin dose×5 doses, n=4). Intravenous (IV) injections were performed once a day for 5 days (on Days 1, 2, 3, 4, 5; total of 5 injections). Dosing began when tumors reached ˜150-250 mm³ in size.

The in vivo experiment was programmed to explore the duration of response after a single cycle. Animals were weighed 3 times a week and the tumor volume quantified. Moreover, animals were monitored daily for signs of pain. Correlation of tumor volume between cell treatments will be examined by Spearman's rank order test.

Dose of curcumin plus doxorubicin micelles was 2.5 mg/kg/day of Dox and 11 mg/kg/day to 32 mg/kg/day of Cur administered IV daily for 5 days. A weekly dosing schedule was also explored, where curcumin plus doxorubicin micelles Dox dose was 8 mg/kg IV weekly.

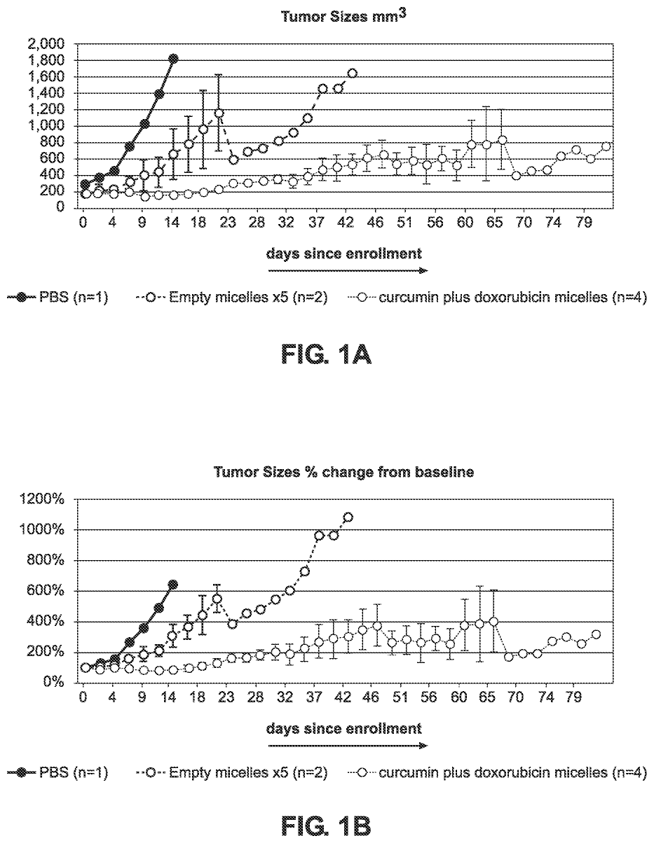

Administration of curcumin plus doxorubicin micelles once a day for 5 days resulted in a reduction in tumor size compared to PBS- and empty micelle-treated mice (FIG. 1A shows tumor sizes in mm³; FIG. 1B shows tumor size percentage change from baseline). Tumor shrinkage was not observed in the PBS controls.

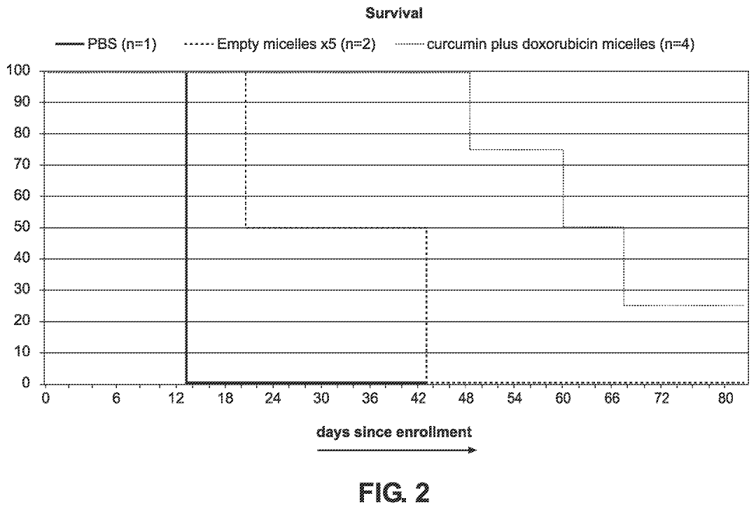

Additionally, an extension of survival time was achieved in curcumin plus doxorubicin micelle-treated mice compared with PBS- and empty micelle-treated mice (FIG. 2).

Overall, IV administration of curcumin plus doxorubicin micelles demonstrated a reduction in tumor size and survival benefit in an STS xenograft mouse model. Thus, the in vivo efficacy data from an animal model in a related disease (a human STS xenograft mouse model) supports the use of curcumin plus doxorubicin micelles for the treatment of Rhabdomyosarcoma (RMS), a rare pediatric disease.

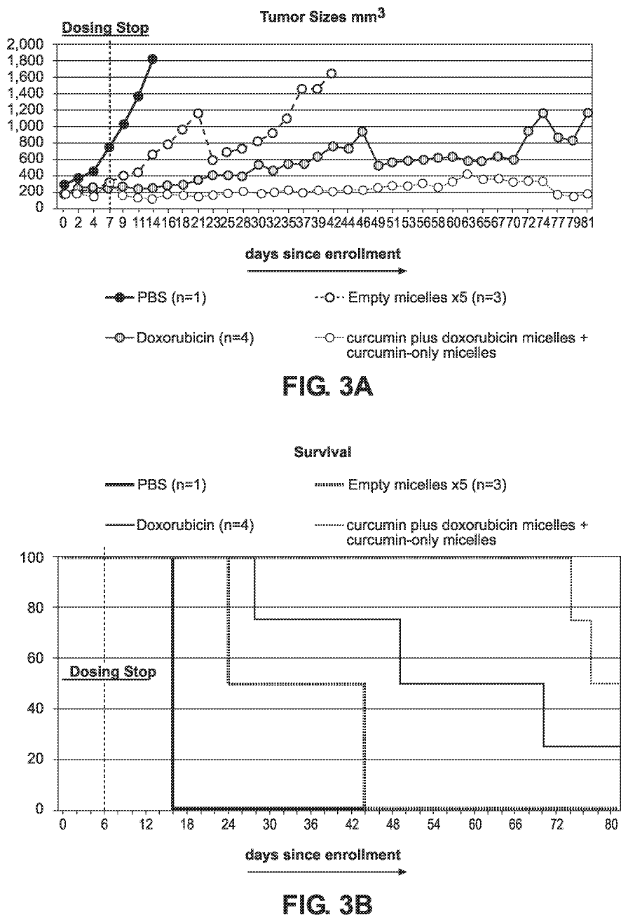

In the same xenograft model of STS, administration of the combination of Curcumin-only micelles with curcumin plus doxorubicin micelles demonstrated further tumor growth suppression (FIG. 3A) that translated into an extended survival (FIG. 3B) without any added toxicity as demonstrated by the lack of weight loss in the treated animals.

In this experimental arm, curcumin-only micelles were administered IV by rapid infusion at the dose of 21.7 mg/kg/dose, followed by curcumin plus doxorubicin micelles administered IV several minutes later. All treated animals received five (5) injections (once a day) at the aforementioned doses, with curcumin-only micelles always being administered prior to curcumin plus doxorubicin micelles. One cycle of five (5) injections of the curcumin-only micelles+curcumin plus doxorubicin micelles combination was administered in the present study.

An administration schedule can additionally include higher doses of curcumin-only micelles, such as 22 to 30 mg/kg/dose, 30 to 50 mg/kg/dose and 50 to 100 mg/kg/dose of curcumin-only micelles. The schedule of administration can include curcumin-only micelles preceding curcumin plus doxorubicin micelles from several minutes to up to 8-12 hours. Curcumin-only micelles can be administered up to eight (8) hours after curcumin plus doxorubicin micelles.

### Example 2

### Effects of Additional Curcumin-Only Micelles and the Duration of Treatment in a Xenograft Model of Sarcoma HT1080

The effect of adding the curcumin-only micelles following curcumin+doxorubicin co-loaded micelles, as well as comparing three (3) days of treatment versus four (4) days of treatment versus five (5) days of treatment was studied in a well-characterized xenograft HT1080 model. HT1080 is a well-characterized fibrosarcoma model. Dodd RD, Mito JK, Kirsch DG. Animal models of soft-tissue sarcoma. Dis Model Mech. 2010 September-October; 3 (9-10): 557-66.

Briefly, HT1080 cells were obtained from ATCC. The HT1080 model was generated by grafting 5×10\hat{ }6 cells subcutaneously (SC) into the left flank of 6- to 8-week-old Nu/Nu mice to develop highly proliferative tumors. The growth of SC tumors was followed by twice weekly measurement of tumor diameters with a Vernier caliper and tumor volume (TV) calculated according to the formula:

$$TV ( {mm}^{3} ) = d 2 \times D / 2$$

where “d” and “D” were the shortest and the longest diameter, respectively.
   All treatments were administered via an intraperitoneal (IP) injection.

Mice were divided into the following treatment groups:
3D IMX-110 Group—curcumin plus doxorubicin micelles (2.5 mg/kg/doxorubicin dose once a day for 3 days) (n=4).
5D IMX-110 Group—curcumin plus doxorubicin micelles (2.5 mg/kg/doxorubicin dose once a day for 5 days) (n=4).
5D IMX-1100-low Group—curcumin plus doxorubicin micelles (2.5 mg/kg/doxorubicin dose once a day for 5 days) followed by curcumin-only micelles (20 mg/kg curcumin/dose) once a day×5 doses (n=4).
5D IMX-1100-med Group—curcumin plus doxorubicin micelles (2.5 mg/kg/doxorubicin dose once a day for 5 days) followed by curcumin-only micelles (20 mg/kg curcumin/dose) administered immediately after followed by another dose of curcumin-only micelles (20 mg/kg curcumin/dose) 1 hour later once a day×5 doses (n=4).
1D-6 IMX-109 Group—curcumin plus doxorubicin micelles (2.5 mg/kg/doxorubicin dose given once) followed by curcumin-only micelles (20 mg/kg curcumin/dose) administered immediately after followed by another five (5) doses of curcumin-only micelles (20 mg/kg curcumin/dose) administered 1 hour apart (n=4).

Dosing began when tumors reached ˜150-250 mm³ in size. The in vivo experiment was programmed to explore the tumor growth inhibition differences between treatment groups. Specifically, the effect of the additional curcumin-only micelles added to curcumin+doxorubicin micelles as well as the difference between one (1) day of dosing versus three (3) days of treatment versus five (5) days of treatment were investigated. Animals were weighed two (2) times per week and the tumor volume quantified. Moreover, animals were monitored daily for signs of pain.

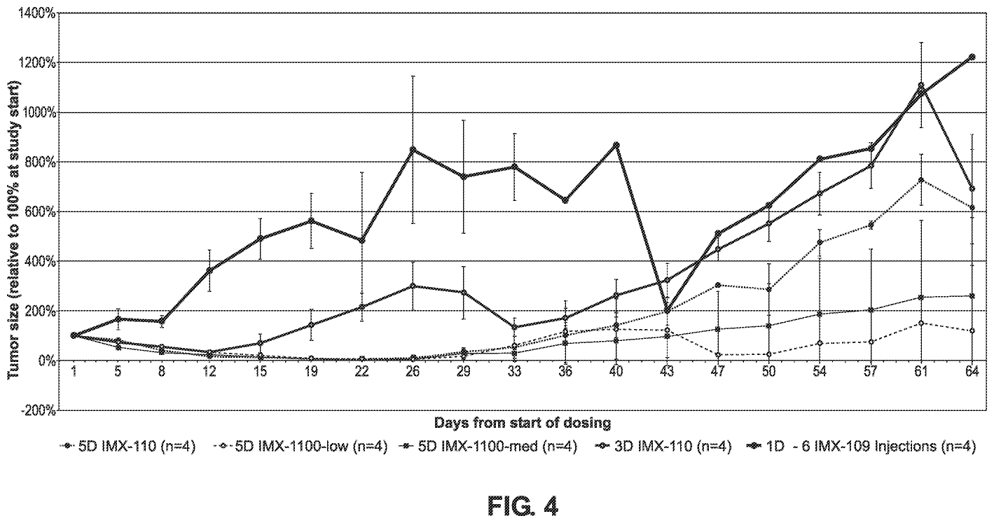

The results of this study demonstrated significantly better tumor growth inhibition with five (5) days of treatment versus three (3) days for up to sixty-four (64) days of observation. Furthermore, the experimental groups that received additional curcumin-only micelle daily doses (5D IMX-1100-low and 5D IMX-1100-med) demonstrated more effective tumor growth inhibition than the curcumin+doxorubicin group 5D IMX-110 (FIG. 4). Notably, the group that received one (1) day of dosing with curcumin+doxorubicin micelles followed by six (6) doses of curcumin-only micelles on the same day showed the weakest tumor growth inhibition compared to the other groups.

An administration schedule can additionally include higher doses of curcumin-only micelles, such as 22 to 30 mg/kg/dose, 30 to 50 mg/kg/dose and 50 to 100 mg/kg/dose of curcumin-only micelles. The schedule of administration can include curcumin-only micelles preceding or following the curcumin plus doxorubicin micelles from several minutes to up to 8-12 hours.

### Example 3

### Effects of Additional Curcumin-Only Micelles and the Dosing Schedules in a Xenograft Model of Sarcoma HT1080

The effect of adding the curcumin-only micelles following curcumin+doxorubicin co-loaded micelles, as well as comparing four (4) days of daily treatment versus five (5) days of daily treatment versus six (6) doses given on Monday, Wednesday, and Friday over two (2) consecutive weeks was studied in a mouse xenograft HT1080 model.

The HT1080 model was generated by grafting 5×10\hat{ }6 cells subcutaneously (SC) into the left flank of 6 to 8 week-old CRL Nu/J mice to develop highly proliferative tumors. The growth of SC tumors was followed by twice weekly measurement of tumor diameters with a Vernier caliper and tumor volume (TV) calculated according to the formula:

$$TV ( {mm}^{3} ) = d 2 \times D / 2$$

where “d” and “D” were the shortest and the longest diameter, respectively.

All treatments were administered via an intraperitoneal (IP) injection.

Mice were divided into the following treatment groups:
PBS Control Group (n=5).
4D-IMX-110 Group—curcumin plus doxorubicin micelles (1.5 mg/kg/doxorubicin dose once a day for 4 days) (n=8).
4D-IMX-1100-low Group—curcumin plus doxorubicin micelles (1.5 mg/kg/doxorubicin dose once a day for 4 days) followed by curcumin-only micelles ([20] mg/kg curcumin/dose) once a day×4 doses (n=8).
4D-IMX-1100-high Group—curcumin plus doxorubicin micelles (1.5 mg/kg/doxorubicin dose once a day for 4 days) followed by curcumin-only micelles ([20] mg/kg curcumin/dose) administered immediately after followed by another two (2) doses of curcumin-only micelles ([20] mg/kg curcumin/dose) 1 hour apart (3 total curcumin-only micelles doses per day) (n=8).
5D-IMX-110 Group—curcumin plus doxorubicin micelles (1.5 mg/kg/doxorubicin dose given once a day for 5 days) (n=4).
5D-IMX-1100-low Group—curcumin plus doxorubicin micelles (1.5 mg/kg/doxorubicin dose given once a day for 5 days) followed by curcumin-only micelles ([20] mg/kg curcumin/dose) once a day×5 days (n=8)
5D-IMX-1100-high Group—curcumin plus doxorubicin micelles (1.5 mg/kg/doxorubicin dose given once a day for 5 days) followed by curcumin-only micelles ([20] mg/kg curcumin/dose) immediately after with two (2) additional doses given 1 hour apart (total 3 curcumin-only doses/day×5 days (n=8).
6D-2W-MWF-1100 Group—curcumin plus doxorubicin micelles (1.5 mg/kg/doxorubicin dose followed by three (3) injections of curcumin-only micelles (20 mg/kg curcumin/dose) given on Monday, Wednesday and Friday of two consecutive weeks for total of 6 days of dosing.

Dosing began when tumors reached ˜150-250 mm³ in size.

The in vivo experiment was programmed to explore the tumor growth inhibition differences between treatment groups. Specifically, the effect of the additional curcumin-only micelles added to curcumin+doxorubicin micelles as well as the difference between four (4) days of dosing versus five (5) days versus six (6) doses administered on Monday, Wednesday and Friday of two consecutive weeks were investigated. Animals were weighed two (2) times a week and the tumor volume quantified. Moreover, animals were monitored daily for signs of pain.

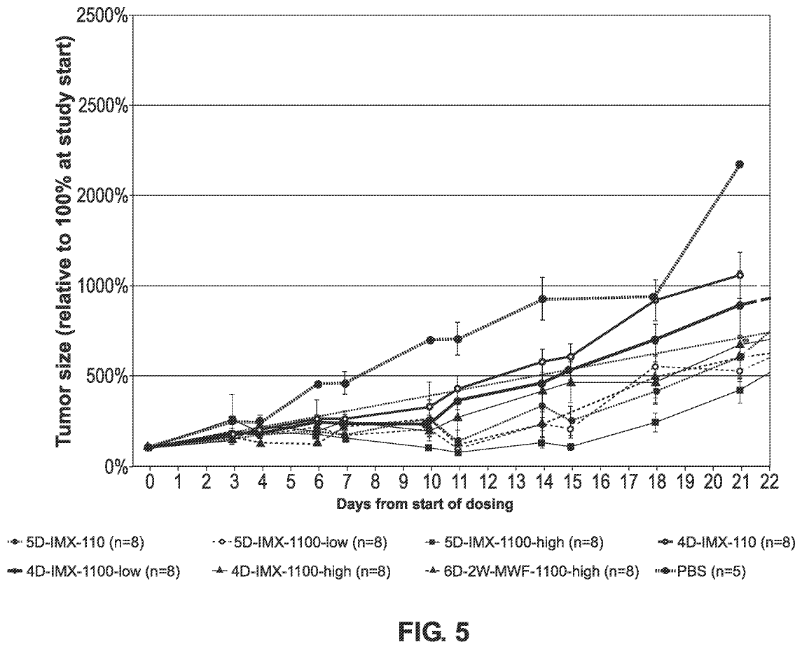

The results of this study demonstrated significantly better tumor growth inhibition with five (5) days of treatment versus four (4) days and versus six (6) doses administered on Monday, Wednesday and Friday of two consecutive weeks up to day twenty-one (21) post dosing. Furthermore, the experimental groups that received additional curcumin-only micelle doses (both, 5-day and 4-day treatment groups) demonstrated more effective tumor growth inhibition than the curcumin+doxorubicin group (FIG. 5). Notably, the group that received six (6) doses over two (2) weeks did not show improved tumor control.

### Example 4

### Effect of Increasing the Number of Daily Doses of Polykinase Inhibitor Curcumin Micelle in a Syngeneic Mouse Sarcoma Model

The effect of increasing the number of daily doses of curcumin micelles from once a day to twice a day to three times a day administered after the co-loaded doxorubicin+curcumin micelle was studied in a syngeneic sarcoma mouse model S180. The sarcoma mouse model S180 is a well-characterized syngeneic mouse sarcoma model. Alfaro G, Lomeli C, Ocadiz R, Ortega V, Barrera R, Ramirez M, Nava G. Immunologic and genetic characterization of S180, a cell line of murine origin capable of growing in different inbred strains of mice. Vet Immunol Immunopathol. 1992 Jan. 31; 30 (4): 385.

The submaximal dose of doxorubicin (1.5 mg/kg) in the curcumin plus doxorubicin micelle was used in this study in order to observe the relative differences in tumor growth inhibition between the experimental groups receiving additional curcumin-only micelles once a day (QD), twice a day (BID or three times a day (TID).

Briefly, S180 cells were obtained from ATCC. The S180 model was generated by grafting 5×10\hat{ }6 cells subcutaneously (SC) into the left flank of 6- to 8-week-old Balb/C mice to develop highly proliferative tumors. The growth of SC tumors was followed by twice weekly measurement of tumor diameters with a Vernier caliper and tumor volume (TV) calculated according to the formula:

$$TV ( {mm}^{3} ) = d 2 \times D / 2$$

where “d” and “D” were the shortest and the longest diameter, respectively.

Mice were divided into the following treatment groups:
Control Group-treated with PBS buffer only (83 μl/dose, n=8);
QD Group—curcumin plus doxorubicin micelles (1.5 mg/kg/doxorubicin dose×5 doses followed by curcumin-only micelles (20 mg/kg curcumin dose) given once a day for 7 days (n=4);
BID Group—curcumin plus doxorubicin micelles (1.5 mg/kg/doxorubicin dose×5 doses) followed by curcumin-only micelles (20 mg/kg curcumin dose) given twice a day for 7 days (n=4);
TID Group—curcumin plus doxorubicin micelles (1.5 mg/kg/doxorubicin dose×5 doses) followed by curcumin-only micelles (20 mg/kg curcumin dose) given three (3) times a day for 7 days (n=4).

All doses were administered by intraperitoneal (IP) injections. Dosing began when tumors reached ˜150-250 mm³ in size. The in vivo experiment was programmed to explore the tumor growth inhibition and the duration of response after a single cycle. Animals were weighed 2 times a week and the tumor volume quantified. Moreover, animals were monitored daily for signs of pain.

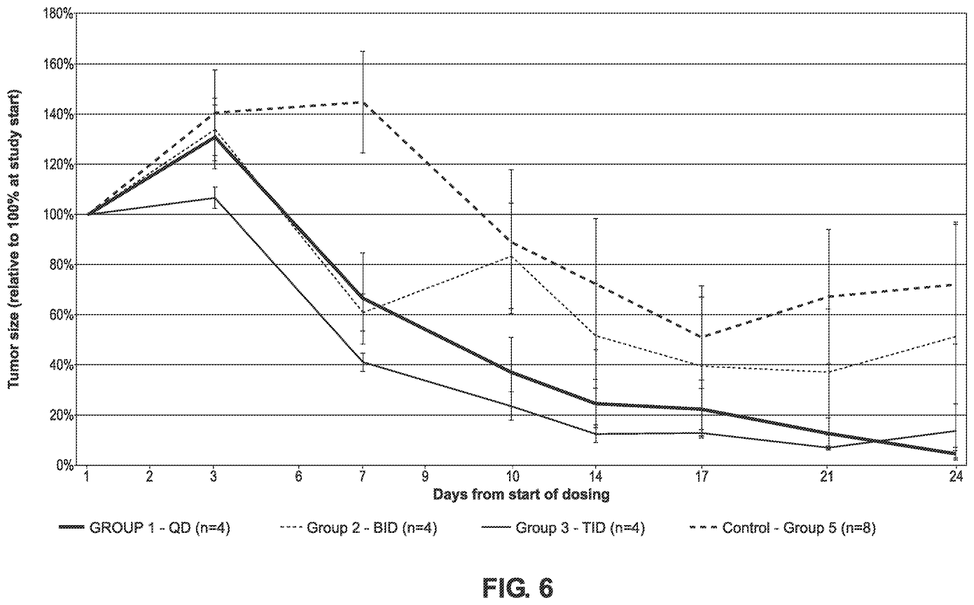

The results of this study demonstrated the increasing tumor growth inhibition with the additional daily doses of curcumin micelles, wherein the tumor control was greater in the TID group than the BID group (FIG. 6) up to 24 days of observation.

Furthermore, there was a statistically significant reduction in variance observed in tumor sizes in the TID group, with much larger variation among animals in the BID and QD groups, suggesting that the increased number of doses of curcuminoid micelles per day resulted in a more uniform and consistent tumor growth suppression.

An administration schedule can additionally include higher doses of curcumin-only micelles, such as 22 to 30 mg/kg/dose, 30 to 50 mg/kg/dose and 50- to 100 mg/kg/dose of curcumin-only micelles. The schedule of administration can include curcumin-only micelles preceding or following the curcumin plus doxorubicin micelles from several minutes to up to 8-12 hours.

### Example 5

Effect of Extending the Treatment Duration with Polykinase Inhibitor Curcumin Micelle in a Syngeneic Mouse Sarcoma Model S180

The effect of extending the treatment course with three (3) per day doses of curcumin micelles to fourteen (14) days was studied in a syngeneic sarcoma mouse model S180. Again, the submaximal dose of doxorubicin (1.5 mg/kg) in the curcumin plus doxorubicin micelle was used in this study in order to observe the difference in tumor growth inhibition contributed by the extended (14 day) treatment schedule with curcumin-only micelles.

Briefly, S180 cells were obtained from ATCC. The S180 model was generated by grafting 5×10\hat{ }6 cells subcutaneously (SC) into the left flank of 6- to 8-week-old Balb/C mice to develop proliferative tumors. The growth of SC tumors was followed by twice weekly measurement of tumor diameters with a Vernier caliper and tumor volume (TV) calculated according to the formula:

$$TV ( {mm}^{3} ) = d 2 \times D / 2$$

where “d” and “D” were the shortest and the longest diameter, respectively.

Dosing began when tumors reached ˜150-250 mm³ in size. Mice were treated for the first five (5) days with curcumin plus doxorubicin micelles (IMX-110-DOX/CUR (1.5 mg/kg/doxorubicin dose for 5 days) plus three (3) doses of curcumin-only micelles IMX-110-CUR (20 mg/kg curcumin dose) given one hour apart, followed by nine (9) days of treatment with three (3) daily doses of IMX-110-CUR (20 mg/kg curcumin dose) given one hour apart (n=4).

All doses were administered by intraperitoneal (IP) injections. The in vivo experiment was programmed to explore the tumor growth inhibition and the duration of response after a single cycle. Animals were weighed 2 times a week and the tumor volume quantified. Moreover, animals were monitored daily for signs of pain.

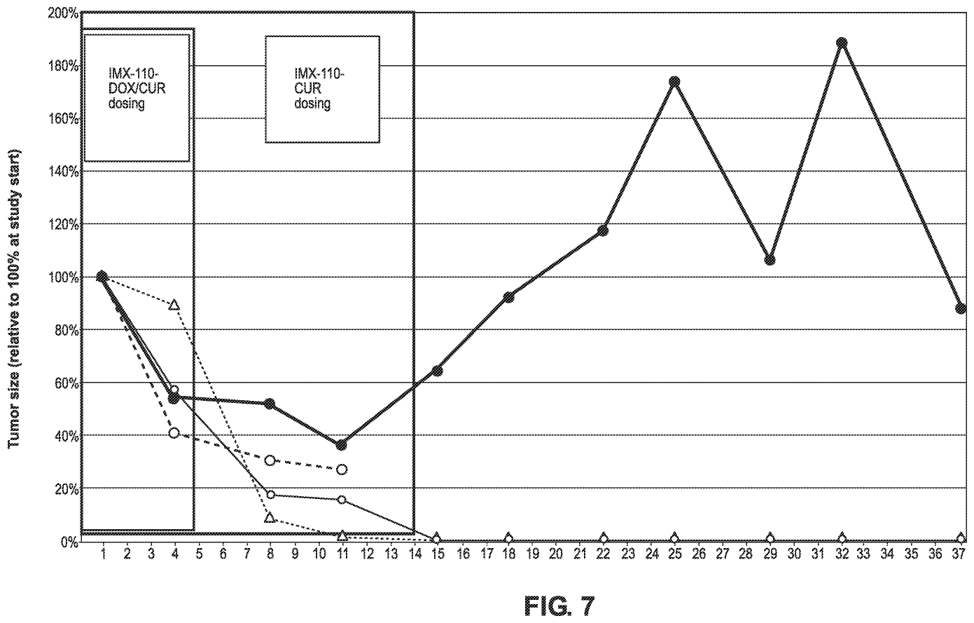

Remarkably, the results of this study demonstrated that 50% of animals (2 out of 4) had complete response to treatment with tumors shrinking below the measurable threshold by day 15. This effect persisted for thirty-seven (37) days of observation highlighting the effect of the 14-day extended treatment schedule with curcumin-only micelles following the combined curcumin plus doxorubicin micelle treatment (FIG. 7).

An administration schedule can additionally include higher doses of curcumin-only micelles, such as 22 to 30 mg/kg/dose, 30 to 50 mg/kg/dose and 50 to 100 mg/kg/dose of curcumin-only micelles. The schedule of administration can include curcumin-only micelles preceding or following the curcumin plus doxorubicin micelles from several minutes to up to 8-12 hours with treatment duration extended to 28 days.

### Example 6

Curcumin Plus Doxorubicin Micelles Human Data in Soft Tissue Sarcoma from the Ongoing Phase 1b/2a Clinical Trial

Curcumin plus doxorubicin micelles are being studied in the ongoing Phase 1b/2a clinical trial in advanced solid tumors. (Clinicaltrials.gov reference: NCT03382340).

Four patients with advanced soft tissue sarcoma were enrolled and dosed with curcumin plus doxorubicin micelles. Data are detailed below.

The first sarcoma patient (#004-001) was a 66-year-old woman with a relapsed Stage IV high grade carcinosarcoma (Grade 4 undifferentiated). Prior to enrollment, her disease progressed after treatment with carboplatin and paclitaxel, manifesting as enlarging pulmonary metastases. Upon enrollment into the curcumin plus doxorubicin micelles trial, the sum of diameters of her target tumor lesions was 109 mm. After the initial two (2) cycles of curcumin plus doxorubicin micelles, her 8-week CT scan showed that disease remained stable with the sum of target lesions at 118 mm at two (2) months. She then underwent an additional two (2) cycles of curcumin plus doxorubicin micelles. The 16-week CT scan demonstrated tumor progression with the sum of target lesions at 137 mm and she was taken off the trial at 4 months. The patient did not experience any drug-related serious adverse events. The clinical benefit observed for this patient was progression-free survival and stable disease for two (2) months.

The second sarcoma patient (#004-002) was a 77-year-old man diagnosed with Stage IV leiomyosarcoma of poorly differentiated Grade 3 histology in 2012 whose tumor relapsed in 2016. Upon the initial diagnosis in 2012, he was treated with Ifosfamide, Gemcitabine, Doxorubicin and Docetaxel. His tumor relapsed in 2016 and he subsequently received Yondelis, Opdivo, Apatinib and Opdivo again. Despite these treatments, the multiple lung metastases and a spleen mass increased in size, and he was enrolled into the curcumin plus doxorubicin micelles trial. At enrollment the sum of diameters of his target tumor lesions was 87 mm. After the initial two (2) cycles of curcumin plus doxorubicin micelles, his 8-week CT scan showed reduction in target tumor sizes by 17% to 72 mm, stable disease by RECIST 1.1 criteria at 2 months. Subsequently, the patient underwent two (2) additional cycles of curcumin plus doxorubicin micelles, and his 16-week CT scan indicated stable disease with sum of diameters of his target tumor lesions being 92 mm at 4 months. However, because of the poor overall health status, the patient elected not to proceed with further treatments. The patient did not experience any drug-related serious adverse events. The clinical benefit observed for this patient was progression-free survival and stable disease for four (4) months.

The third sarcoma patient (#004-003) was a 59-year-old man with Stage IV leiomyosarcoma of poorly differentiated Grade 3 histology. After his diagnosis in 2017, prior to enrollment in the curcumin plus doxorubicin micelles trial, the patient received Gemcitabine, Docetaxel, Ifosfamide, Dacarbazine, Doxorubicin, Olarutumab, Yondelis, Nivolumab, Yondelis again, Nivolumab again, and TVEC. Upon enrollment into the curcumin plus doxorubicin micelles trial, the sum of diameters of his target tumor lesions was 312 mm. After the initial two (2) cycles of curcumin plus doxorubicin micelles, the 8-week CT scan showed Stable Disease with reduction of the sum of target lesions to 306 mm at 2 months. The 16-week CT scan after four (4) cycles of curcumin plus doxorubicin micelles again showed Stable Disease with the sum of target lesions of 313 mm at 4 months. The 24-week CT scan after six (6) cycles of curcumin plus doxorubicin micelles again demonstrated Stable Disease with the reduction of the sum of target lesions to 257 mm, a reduction of 18% from baseline at 6 months. At this point, this patient had received the cumulative dose of 733 mg/m² of Doxorubicin and was taken off the trial for further monitoring. The patient did not experience any drug-related serious adverse events. The clinical benefit observed for this patient was progression-free survival and stable disease for six (6) months.

The fourth patient with sarcoma (#004-005) was a 27-year-old woman with Stage IV poorly differentiated Grade 3 sarcoma who progressed after Yondelis, Opdivo and Ipilumomab with enlarging pulmonary and extrapulmonary intrathoracic metastases. Upon enrollment into the curcumin plus doxorubicin micelles trial, the sum of diameters of her target tumor lesions was 94 mm. After the initial two (2) cycles of curcumin plus doxorubicin micelles, the 8-week CT scan showed Stable Disease with the sum of target lesions of 94 mm at 2 months. The 16-week CT scan after four (4) cycles of curcumin plus doxorubicin micelles again showed Stable Disease with the sum of target lesions decreasing to 85 mm at 4 months. The 24-week CT scan after six (6) cycles of curcumin plus doxorubicin micelles showed the sum of the target lesions was 111 mm (18% increase vs. baseline) at 6 months. Despite not reaching the 20% threshold of progression by RECIST 1.1 criteria, the patient was deemed as having clinical progression and was taken off trial. The patient did not experience any drug-related serious adverse events. The clinical benefit observed for this patient was progression-free survival and stable disease for four (4) months.

In summary, curcumin plus doxorubicin micelles was administered to four (4) patients with relapsed/refractory soft tissue sarcoma who progressed after three (3) or more prior lines of treatment in its Phase 1/2a trial. Not a single patient progressed after the first two (2) cycles, with all four (4) showing disease control at two (2) months. Notably, patient #004-003 experienced 6-month progression free survival, despite having the largest tumor burden at the time of enrollment. Further, patients #004-002 and #004-005 experienced 4-month progression free survival. No patient experienced drug-related SAEs.

The various methods and techniques described above provide a number of ways to carry out the application. Of course, it is to be understood that not necessarily all objectives or advantages described are achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by including one, another, or several other features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, any numbers expressing quantities of ingredients, properties such as molecular weight, reaction conditions, and so forth, used to describe and claim certain embodiments of the disclosure are to be understood as being modified in some instances by the term “about.” Accordingly, in some embodiments, the numerical parameters set forth in the written description and any included claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are usually reported as precisely as practicable.

In some embodiments, the terms “a” and “an” and “the” and similar references used in the context of describing a particular embodiment of the application (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Variations on preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Please amend the claims as indicated below:

1. A pharmaceutical regimen comprising:
a first micelle construct comprising a polykinase inhibitor and a chemotherapeutic agent; and
a second micelle construct comprising and a polykinase inhibitor.

2. The pharmaceutical regimen of claim 1, wherein the second micelle construct does not comprise a chemotherapeutic agent.

3. The pharmaceutical regimen of claim 1, wherein the polykinase inhibitor is selected from a curcuminoid or curcuminoid analog, derivative or salt thereof or combination thereof.

4. The pharmaceutical regimen of claim 3, wherein the wherein the curcuminoid or curcuminoid analog, derivative or salt thereof or combination thereof is a curcumin compound having the structure of formula 1: 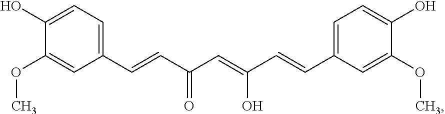 [SMILES: COc1cc(/C=C/C(=O)/C=C(O)/C=C/c2ccc(O)c(OC)c2)ccc1O]
or a curcumin compound having the structure of formula 2:

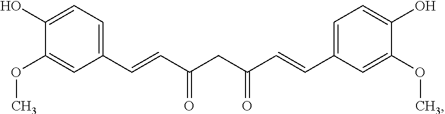 [SMILES: COc1cc(/C=C/C(=O)CC(=O)/C=C/c2ccc(O)c(OC)c2)ccc1O]

5. The pharmaceutical regimen of claim 1, wherein the chemotherapeutic agent is doxorubicin or a pharmaceutical equivalent, analog, derivative, and/or salt thereof.

6. The pharmaceutical regimen of claim 1, wherein the first and second micelle constructs are each formed by amphiphilic PEG2000-DSPE polymers.

7. The pharmaceutical regimen of claim 1, wherein the first and second micelle constructs are each between 10 nm and 20 nm.

8. The pharmaceutical regimen of claim 1, wherein the first and second micelle constructs are each between 20 nm and 60 nm.

9. The pharmaceutical regimen of claim 1, wherein the first and second micelle constructs are each less than 30 nm.

10. (canceled)

11. A method of treating a sarcoma in a subject, comprising administering a therapeutically effective dosage of the regimen of claim 1 to the subject.

12. (canceled)

13. (canceled)

14. The method of claim 11, wherein administration of the second micelle construct comprising a polykinase inhibitor precedes administration of the first micelle construct comprising a polykinase inhibitor and a chemotherapeutic agent.

15. The method of claim 11, wherein administration of the second micelle construct comprising a polykinase inhibitor follows administration of the first micelle construct comprising a polykinase inhibitor and a chemotherapeutic agent.

16. The method of claim 11, wherein the second micelle construct comprising a polykinase inhibitor is administered once per day.

17. The method of claim 11, wherein the second micelle construct comprising a polykinase inhibitor is administered twice per day.

18. The method of claim 11, wherein the second micelle construct comprising a polykinase inhibitor is administered three times per day.

19. The method of claim 11, wherein the method further comprises administering the second micelle construct comprising a polykinase inhibitor for up to 14 days after completion of the regimen of claim 1.

20. The method of claim 11, wherein the method further comprises administering the second micelle construct comprising a polykinase inhibitor for 15 to 28 days after completion of the regimen of claim 1.

21. The method of claim 11, wherein the method further comprises administering the second micelle construct comprising a polykinase inhibitor for 28 days or more after completion of the regimen of claim 1.

22. The method of claim 11, wherein the second micelle construct comprising a polykinase inhibitor is administered at a dosage of about 20 mg/m² to about 200 mg/m² per day.

23. A method of inhibiting cell growth of a tumor cell, comprising administering a therapeutically effective dosage of the regimen of claim 1 to the tumor cell.

24. (canceled)

25. (canceled)
